(19) United States
(12) Patent Application Publication
(10) Pub. No.: US 2016/0045183 A1
(43) Pub. Date: Feb. 18, 2016

(54) ELECTRONIC STETHOSCOPE APPARATUS, AUTOMATIC DIAGNOSTIC APPARATUS AND METHOD

(71) Applicant: SAMSUNG ELECTRONICS CO., LTD., Suwon-shi (KR)

(72) Inventors: Yoon-jae LEE, Seoul (KR); Young-tae KIM, Seongnam-si (KR); Seoung-hun KIM, Hwaseong-si (KR); Jong-youb RYU, Suwon-si (KR)

(73) Assignee: SAMSUNG ELECTRONICS CO., LTD., Suwon-si (KR)

(21) Appl. No.: 14/782,467

(22) PCT Filed: Apr. 7, 2014
(86) PCT No.: PCT/KR2014/002951

§ 371 Date: Oct. 5, 2015

Related U.S. Application Data
(60) Provisional application No. 61/815,928, filed on Apr. 25, 2013.
(60) Provisional application No. 61/808,699, filed on Apr. 5, 2013.

(30) Foreign Application Priority Data
Apr. 4, 2014 (KR) 10-2014-0040719

(51) Int. Cl. A61B 7/04; A61B 8/08; A61B 8/14

(52) U.S. Cl. CPC A61B 7/04; A61B 8/14; A61B 8/5207; A61B 8/5223

(57) ABSTRACT

An electronic stethoscope apparatus includes a bioacoustics sensor configured to sense bioacoustics; a noise sensor configured to sense noise in the sensed bioacoustics; and a noise remover configured to remove the sensed noise and output the bioacoustics from which the noise has been removed.

### CROSS-REFERENCE TO RELATED APPLICATIONS

This application is a U.S. national stage application under 35 USC 371 of International Application No. PCT/KR2014/002951, filed on Apr. 7, 2014, in the Korean Intellectual Property Office, which claims the benefit of U.S. Provisional Application Nos. 61/808,699, filed on Apr. 5, 2013, and 61/815,928, filed on Apr. 25, 2013, in the United States Patent and Trademark Office, and priority from Korean Patent Application No. 10-2014-0040719, filed on Apr. 4, 2014, in the Korean Intellectual Property Office, the disclosures of which are incorporated herein in their entireties by reference.

### BACKGROUND

1. Technical Field

Apparatuses and methods consistent with exemplary embodiments relate to an electronic stethoscope apparatus, and more particularly, to an electronic stethoscope apparatus which detects bioacoustics by efficiently removing noise, an automatic diagnostic apparatus and method.

2. Description of Related Art

Since an electronic stethoscope apparatus uses a microphone to detect bioacoustics, noise may be also detected according to the sensitivity of the microphone. In particular, noise is likely to occur when a chest piece is rubbed against the body, and extraneous noise of the chest piece may be detected.

The occurrence of noise affects the accuracy of diagnosis by using the electronic stethoscope apparatus, and thus, a method of removing noise properly is needed.

Meanwhile, bioacoustics has different signal features according to the body part and it is possible to diagnose disease when analyzing the signal features of the bioacoustics. In addition, while diseases have been diagnosed by separately using an ultrasonic device, a pulse wave device, and an electrocardiography device, it is possible to perform a more detailed diagnosis of a disease by using a combination of several diagnostic devices. Therefore, there is a need for a technique for measuring bioacoustics and diagnosing diseases by considering information from different diagnostic devices.

### SUMMARY

One or more exemplary embodiments provide an electronic stethoscope apparatus which can remove noise properly.

One or more exemplary embodiments also provide a technique for measuring bioacoustics and diagnosing diseases by considering information from other devices.

According to an aspect of an exemplary embodiment, provided is an electronic stethoscope apparatus including: a bioacoustics sensor configured to sense bioacoustics of an object; a noise sensor configured to sense a noise in the sensed bioacoustics; and a noise remover configured to remove the sensed noise and output the sensed bioacoustics from which the noise has been removed.

The bioacoustics sensor may include a microphone.

The noise sensor may include a microphone and mounted on a chest piece of the electronic stethoscope apparatus, and the noise remover may be configured to output the sensed bioacoustics by filtering a frequency of a noise signal sensed by the microphone.

The noise sensor may include a movement sensor configured to sense movement of the electronic stethoscope apparatus, and the noise remover may be configured to output the sensed bioacoustics by filtering a frequency of a noise signal sensed by the movement sensor.

According to an aspect of another exemplary embodiment, provided is an electronic stethoscope apparatus including: a bioacoustics sensor configured to sense bioacoustics of an object; an electrocardiography (ECG) detector configured to detect an electrocardiography signal from the object; and a noise remover configured to estimate a position of a heart sound from the sensed bioacoustics by using the detected electrocardiography signal and remove a corresponding noise from the sensed bioacoustics based on the position of the heart sound.

The bioacoustics sensor may be configured to extract a feature of the detected electrocardiography (ECG) signal, and the noise remover may be configured to estimate the position of the heart sound from the sensed bioacoustics by using the feature of the detected electrocardiography (ECG) signal.

According to an aspect of still another exemplary embodiment, provided is an electronic stethoscope apparatus including: a bioacoustics sensor configured to sense bioacoustics of an object; a pulse wave signal detector configured to detect a pulse wave signal of the object; and a pulse wave velocity calculator configured to calculate a pulse wave transfer velocity by using a distance between the bioacoustics sensor and the pulse wave signal detector and a time difference between detecting a first position of the sensed bioacoustics and detecting a second position of the detected pulse wave signal.

According to an aspect of still another exemplary embodiment, provided is an automatic diagnosis apparatus including: a bioacoustics sensor configured to sense bioacoustics of an object; an ultrasonic image generator configured to generate an ultrasonic image by irradiating an ultrasonic signal onto the object and sensing the ultrasonic signal that is reflected from the object; and an automatic diagnoser configured to diagnose a disease by using the sensed bioacoustics and the generated ultrasonic image.

According to an aspect of still another exemplary embodiment, provided is an automatic diagnosis method by using an electronic stethoscope apparatus, the automatic diagnosis method including: sensing a bioacoustics of an object; sensing a noise in the sensed bioacoustics; and removing the sensed noise and output the sensed bioacoustics from which the noise has been removed.

The automatic diagnosis method may further include providing a microphone on a chest piece of the electronic stethoscope apparatus, wherein the sensing the noise is performed by using the microphone.

The removing may include removing the sensed noise from the sensed bioacoustics by filtering a frequency of a noise signal sensed by the microphone.

The automatic diagnosis method may further include providing a movement sensor configured to sense movement of the electronic stethoscope apparatus, wherein the sensing the noise is performed by the movement sensor, wherein the removing includes removing the sensed noise from the sensed bioacoustics by filtering a frequency of a noise signal that is sensed based on the movement sensor.

According to an aspect of still another exemplary embodiment, provided is an automatic diagnosis method including: sensing bioacoustics of an object; detecting an electrocardiography signal of the object; estimating a position of a heart sound from the sensed bioacoustics by using the detecting electrocardiography signal and removing a corresponding noise from the sensed bioacoustics based on the position of the heart sound.

The detecting may include extracting a feature of the detected electrocardiography signal, and the estimating includes estimating the position of the heart sound from the sensed bioacoustics by using the extracted feature of the detected electrocardiography signal.

According to an aspect of still another exemplary embodiment, provided is an automatic diagnosis method including: sensing bioacoustics of an object; detecting a pulse wave signal of the object; and calculating a pulse wave transfer velocity by using a distance in locations of the sensing and the detecting and a time difference between detecting a first position of the sensed bioacoustics and detecting a second position of the detected pulse wave signal.

### BRIEF DESCRIPTION OF DRAWINGS

The above and/or other aspects will be more apparent by describing certain example embodiments with reference to the accompanying drawings.

FIG. 1 is a block diagram illustrating a configuration of an electronic stethoscope apparatus according to an exemplary embodiment.

FIGS. 2, 3, 4, and 5 are block diagrams illustrating a configuration of an electronic stethoscope apparatus according to various exemplary embodiments.

FIG. 6 is a reference diagram illustrating a method of separating target bioacoustics from bioacoustics where a plurality of source bioacoustics are synthesized.

FIG. 7 is a block diagram illustrating a configuration of an electronic stethoscope apparatus according to another exemplary embodiment.

FIG. 8 is a block diagram of a configuration of an electronic stethoscope apparatus according to another exemplary embodiment.

FIG. 9 is a block diagram illustrating a block diagram illustrating a configuration of an electronic stethoscope apparatus according to another exemplary embodiment.

FIG. 10 is a reference diagram schematically illustrating an operation of an electronic stethoscope apparatus according to another exemplary embodiment.

FIG. 11 is a drawing illustrating a wave form of an electrocardiography signal.

FIG. 12 is a drawing illustrating a location correlation between a stethoscope signal and an electrocardiography signal.

FIG. 13 is a block diagram illustrating a configuration of an electronic stethoscope apparatus according to another exemplary embodiment.

FIG. 14 is a reference diagram schematically illustrating an operation of an electronic stethoscope apparatus according to another exemplary embodiment.

FIG. 15 is a drawing illustrating wave forms of a heart sound and a pulse wave.

FIG. 16 is a block diagram illustrating an operation of an electronic stethoscope apparatus according to another exemplary embodiment.

FIG. 17 is a reference diagram schematically illustrating an operation of an electronic stethoscope apparatus according to another exemplary embodiment.

FIG. 18 is a block diagram illustrating a configuration of a remote diagnosis system according to an exemplary embodiment.

FIG. 19 is a flowchart illustrating a remote diagnosis method according to an exemplary embodiment.

FIGS. 20, 21, 22, and 23 are flowcharts of an automatic diagnostic method according to various exemplary embodiments.

### DETAILED DESCRIPTION

Hereinafter, description will be given in detail of exemplary embodiments with reference to the accompanying drawings.

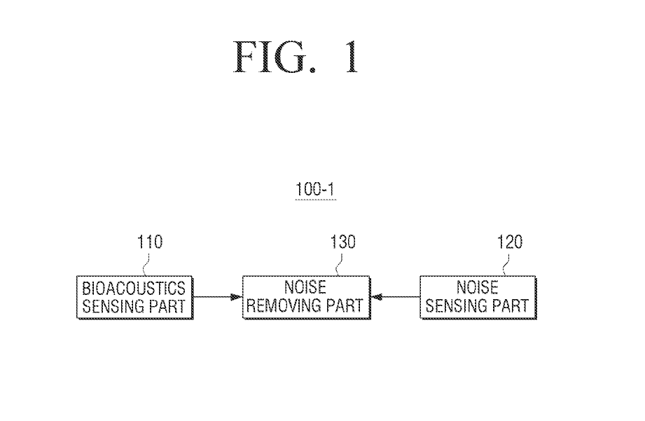

FIG. 1 is a block diagram illustrating a configuration of an electronic stethoscope apparatus 100-1 according to an exemplary embodiment.

Referring to FIG. 1, the electronic stethoscope apparatus 100-1 includes a bioacoustics sensing part (or a bioacoustics sensor) 110, a noise sensing part (or a noise sensor) 120, and a noise removing part (or a noise remover) 130.

The bioacoustics sensing part 110 is for sensing bioacoustics. Specifically, the bioacoustics sensing part 110 uses at least one stethoscope sensor to sense various types of bioacoustics. At this point, the bioacoustics may be at least one sound among a pulmonary sound, a heart sound, and an abdomen sound. Specially, the bioacoustics has different frequency features such as frequency bands and periods etc. For example, a heart sound may have a low frequency and a pulmonary sound may have relatively a low frequency. Also, bioacoustics sensed by the bioacoustics sensing part 110 may be a sound of a combination of various bioacoustics.

According to an exemplary embodiment, the bioacoustics sensing part 110 may include a stethoscope sensor (not illustrated), a sensor driving part (not illustrated), an amplifying part (not illustrated), a filtering part (not illustrated), and an analog-to-digital (A/D) converting part (not illustrated). The stethoscope sensor collects bioacoustics through physical contact and/or physical non-contact. Here, the stethoscope sensor is a sensor configured to collect bioacoustics and convert the collected bioacoustics into an electrical signal, and may include a microphone. The microphone may be a microphone to have physical contact with a body and may comprise an impedance matching circuit using a piezo film. The microphone may be mounted on a chest piece of the stethoscope sensor to have physical contact to collect bioacoustics.

Two or more than two stethoscope sensors may be included to collect bioacoustics from several locations.

The sensor driving part operates a stethoscope sensor and outputs a plurality of bioacoustics signals that are converted into electrical signals by the stethoscope sensor. Therefore, the sensor driving part may include a number of sensor driving modules corresponding to a number of the stethoscope sensors.

The amplifying part amplifies each of a plurality of bioacoustics, which are output from the sensor driving part, to have a desired amplification gain.

The filtering part may remove a high frequency or low frequency noise from collected bioacoustics. The function of the filtering part may be performed by a noise removing part 130 which is to be described later.

The A/D converting part converts the bioacoustics filtered by the filtering part or the noise removing part into digital signals and outputs the converted bioacoustics.

The noise sensing part 120 senses the noise generated in the bioacoustics sensing process. The noise sensing part 120 may include a configuration similar to that of the bioacoustics sensing part 110.

That is, the noise sensing part 120 may include a noise sensing sensor (not illustrated), a sensor driving part (not illustrated), an amplifying part (not illustrated), a filtering part (not illustrated), and an A/D converting part (not illustrated).

The noise sensing sensor is located within or outside the chest piece and may sense a noise.

The noise sensing sensor includes a microphone and may sense noise that is generated by contacting skin in the bioacoustics sensing process. The sensed noise may be output after being properly processed with, for example, amplification, filtering or A/D conversion.

The noise sensing sensor may include a movement sensor. The movement sensor may be located primarily in the chest piece and sense the movement of the chest piece. For example, the movement sensor senses vibration of the chest piece generated in the bioacoustics measuring process.

The movement sensor may be embodied as various sensors such as a gravitational acceleration sensor, a geomagnetic sensor, and a gyro sensor etc.

For example, when the movement sensor is embodied as a fluxgate geomagnetic sensor using a fluxgate, the movement sensor includes a fluxgate core including high permeable magnetic materials such as permalloy, a driving coil that winds around a core, and a fluxgate sensor formed with a detecting coil. The number of the fluxgate core may be two or three, for example. The fluxgate cores may be provided in a mutually orthogonal form. That is, a biaxial fluxgate sensor may be provided by using X-axis and Y-axis fluxgates. A triaxial fluxgate sensor may be provided by using X-axis, Y-axis, and Z-axis fluxgates. Accordingly, the size and the direction of an external magnetic field are measured by detecting a second harmonic component that is proportional to the external magnetic field by using the detecting coil when magnetism is generated in response to an operating signal in each operating coil where each fluxgate core is wound. Therefore, a rotation angle and a rotation direction may be sensed by comparing a direction of a previously measured magnetic field with a direction of a currently measured magnetic field.

For another example, the movement sensor may include a gyro sensor. The gyro sensor is a sensor for measuring an angle that has moved in one second. That is, Coriolis force occurs when an object moves and the gyro sensor uses a formula of Coriolis force to sense an angular speed that works in an inertial frame. Therefore, a rotation angle and a rotation direction may be sensed.

The movement sensor may further include an acceleration sensor to compensate for influence according to a tilting degree. That is, the movement sensor may calculate the exact rotation angle and rotation direction by considering a tilt angle such as a pitch angle and a roll angle that is measured by the acceleration sensor.

The noise removing part 130 removes the noise from the sensed bioacoustics by, for example, using filters h1 and h2, and outputs the sensed bioacoustics from which the noise has been removed.

Specifically, when the noise sensing part 120 includes a microphone, the noise removing part 130 outputs the sensed bioacoustics by filtering the frequency of a noise signal sensed by the microphone. The microphone senses a microphone input background noise signal as a reference signal. Then, the noise removing part 130 uses the reference signal to remove the background noise introduced into the stethoscope sensor by using an adaptation filter.

When the noise sensing part 120 includes a movement sensor for sensing a movement of an electronic stethoscope apparatus 100-1, the noise removing part 130 uses a sensing value which is output from the movement sensor to calculate the noise signal and outputs the sensed bioacoustics by filtering the frequency of the noise signal. When a chest piece moves to a horizontal or vertical direction and comes into contact with a body, the noise occurs, which is sensed by the noise sensing part 120. Then, the noise removing part 130 uses the movement direction and size of the bioacoustics sensing part 110 and an interrelationship among friction noise to remove the noise signal. The interrelationship here means a relationship of the size and the features of the noise that occurs according to the movement direction and the movement size of the bioacoustics sensing part 110.

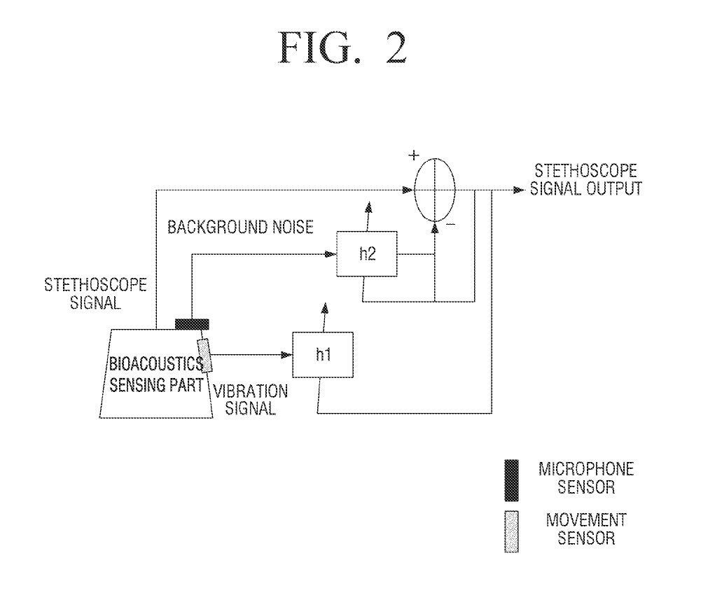

FIGS. 2, 3, 4, and 5 are block diagrams illustrating a configuration of an electronic stethoscope apparatus according to various exemplary embodiments.

Referring to FIG. 2, the electronic stethoscope apparatus 100-1 according to an exemplary embodiment includes a movement sensor which can be mounted on an inner or outer surface of the chest piece. The movement sensor senses the movement of the chest piece while the stethoscope sensor progresses and outputs movement data. The noise removing part 130 uses the movement data to estimate a noise occurring area. In an exemplary embodiment, the noise data corresponding to the accumulated movement data is stored in a storing part (not illustrated) and the measured movement data can be mapped thereto. The noise removing part 130 outputs the sensed bioacoustics by filtering the frequency of the noise signal.

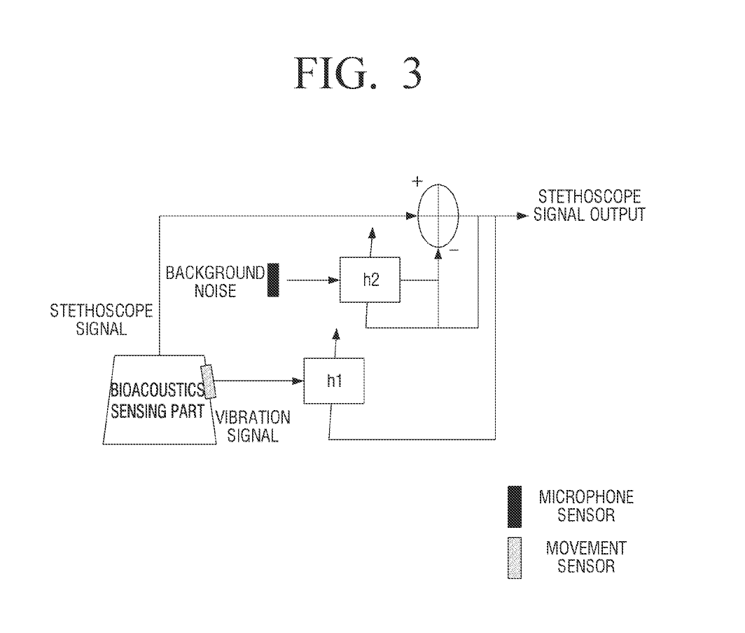

Referring to FIG. 3, the electronic stethoscope apparatus 100-1 according to another exemplary embodiment includes a movement sensor mounted on the inner or outer surface of the chest piece, and the microphone can be located outside of the chest piece. In this case, the movement sensor senses the movement of the chest piece while the stethoscope sensor progresses and outputs the movement data. The noise removing part 130 uses the movement data to estimate the noise occurring area. The noise removing part 130 outputs the sensed bioacoustics by filtering the frequency of the noise signal. The microphone senses and outputs the noise generated in the bioacoustics measuring process from the outside of the chest piece. Then noise removing part 130 outputs the sensed bioacoustics by filtering the frequency of the sensed noise signal with the microphone.

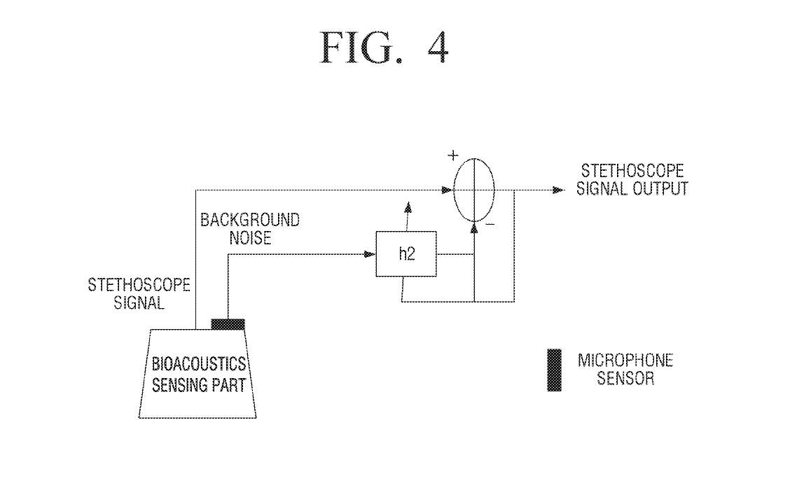

Referring to FIG. 4, the electronic stethoscope apparatus 100-1 according to another exemplary embodiment includes a microphone mounted on the inner or outer surface of the chest piece. In this case, the microphone senses and outputs the noise generated in the bioacoustics measuring process. Then, the noise removing part 130 outputs the sensed bioacoustics by filtering the frequency of the sensed noise signal from the microphone.

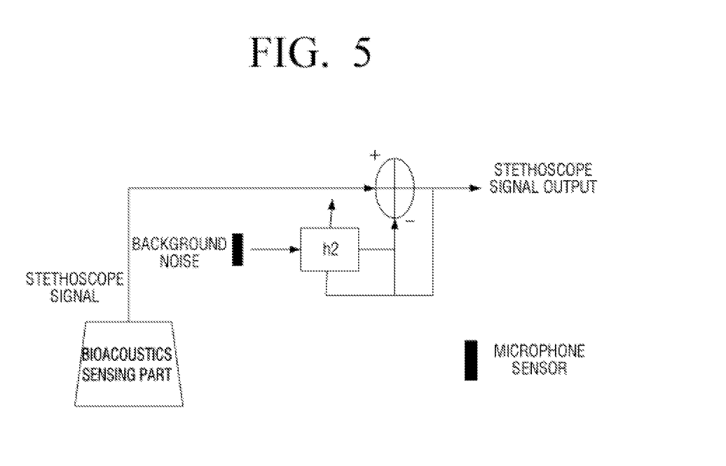

Referring to FIG. 5, the electronic stethoscope apparatus 100-1 according to another exemplary embodiment includes a microphone located outside of the chest piece. In this case, an operation of the electronic stethoscope apparatus 100-1 may be similar to that of the above described exemplary embodiment.

A human body generates bioacoustics having different characteristics from various body parts. For example, a heart sound, a pulmonary sound, and an abdomen sound occur around a human abdomen. In this case, when locating a sensor of a stethoscope apparatus around the abdomen, the stethoscope apparatus senses bioacoustics synthesized with a heart sound, a pulmonary sound and an abdomen sound.

In particular, when a doctor uses a stethoscope apparatus to diagnose the body condition of a person, if the doctor cannot detect exact target bioacoustics, the doctor may misdiagnose based on the bioacoustics that is not detected properly.

Therefore, it is desirable to detect the target bioacoustics from the bioacoustics in which bioacoustics from a plurality of sources (or source bioacoustics) are synthesized such that a doctor may properly diagnose based on the target bioacoustics.

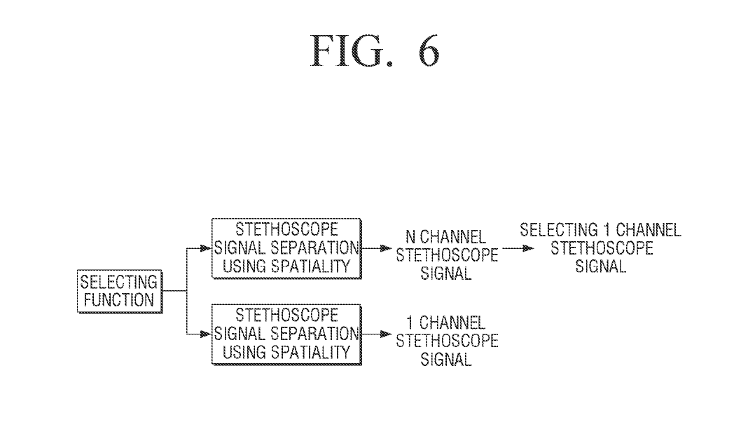

FIG. 6 is a reference diagram illustrating a method of separating target bioacoustics from bioacoustics in which bioacoustics from a plurality of sources is synthesized.

Referring to FIG. 6, the method according to an exemplary embodiment may detect the target bioacoustics for diagnosis by considering the spatiality of the bioacoustics (e.g., n channel stethoscope signal) sensed by a multi stethoscope sensor and separating the source bioacoustics. For example, the method may separate target bioacoustics such that one channel stethoscope signal is selected.

In an exemplary embodiment, the target bioacoustics may be detected by removing reference bioacoustics sensed by a second stethoscope sensor from the bioacoustics sensed by a first stethoscope sensor.

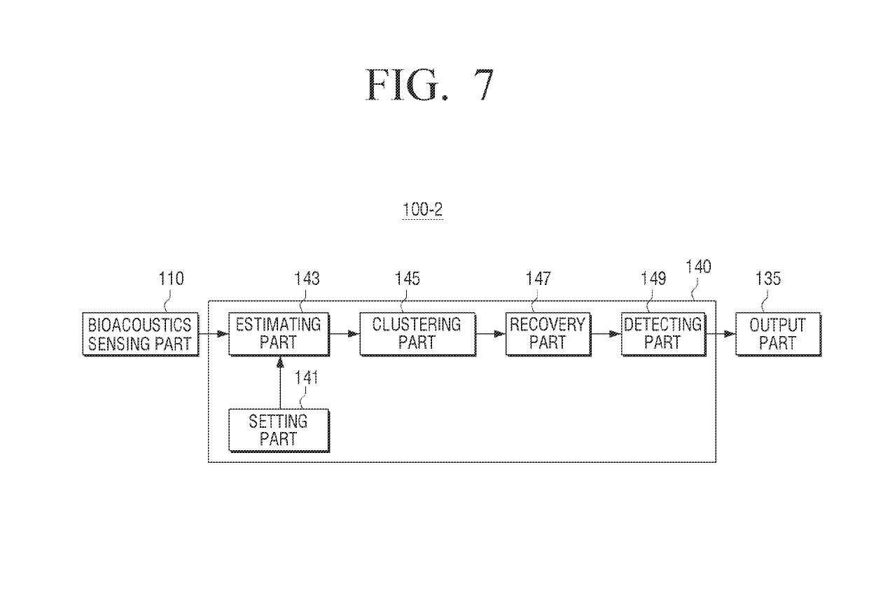

FIG. 7 is a block diagram illustrating a configuration of an electronic stethoscope apparatus 100-2 according to another exemplary embodiment.

Referring to FIG. 7, the electronic stethoscope apparatus 100-2 according to another exemplary embodiment includes a bioacoustics sensing part, 110, a separation part 140, and an output part 135.

The bioacoustics sensing part 110 is as described above, and thus, the description thereof will be omitted.

The separation part 140 uses the spatiality of the bioacoustics to separate a plurality of source bioacoustics from the sensed bioacoustics. The spatiality of the bioacoustics can be represented as a gain and a delay of bioacoustics sensed through a plurality of stethoscope sensors. That is, the separation part 140 may analyze the gain and delay of source bioacoustics and separate a plurality of source bioacoustics from the sensed bioacoustics.

The separation part 140 includes a setting part 141, an estimating part 143, a clustering part 145, a recovery part 147, and a detecting part 149.

The setting part 141 sets a signal mixing model based on spatiality of bioacoustics. For example, the setting part 141 may set the signal mixing model as Equation 1 below when receiving bioacoustics including two source bioacoustics from two stethoscope sensors.

x₁(t)=s₁(1)+s₂(t) <Equation 1>

xₜ(t)=a₁s₁(t−d₁)+a₂s₂(t−d₂)

Here, x1 is a signal received by the stethoscope sensor. s1 and s2 are the first source bioacoustics signal and the second source bioacoustics signal, respectively. a1 and a2 are the gain damping ratios of the first and second source bioacoustics signals, respectively. d1 and d2 are the delay values of the first and the second source bioacoustics signals, respectively.

The estimating part 143 uses the signal mixing model that is set by the setting part 141 to estimate a mixing parameter of a bioacoustics signal received by each stethoscope sensor. The mixing parameter may include the gain damping ratio a and the delay value d.

The clustering part 145 clusters the mixing parameter estimated by the estimating part 143 in a parameter space. Specifically, the clustering part 145 may cluster the estimated mixing parameters in the parameter space having an x-axis as the gain damping ratio a and a y-axis as the delay value d.

The recovery part 147 uses the mixing parameters clustered by the clustering part 145 to convert the bioacoustics into a time domain and recover a plurality of source bioacoustics. For example, the recovery part 147 may convert the signal detected in a first domain from the parameter space into the time domain and recover the signal as the first source bioacoustics, and may convert the signal detected in a second domain into the time domain and recover the signal as the second source bioacoustics.

The detecting part 149 detects the target bioacoustics among a plurality source bioacoustics recovered by the recover part 147. The target bioacoustics may be bioacoustics selected by the user but it is not limited to this, and may be set as default in a manufacturing process.

The stethoscope 100-2 may further include a user input part (not illustrated) for selecting the target bioacoustics to be detected.

The output part 135 may output the detected target bioacoustics. The output part 135 may output the target bioacoustics in an audio form, however, this is only an example and the target bioacoustics may be output in any other forms, for example, a video form.

By using the stethoscope apparatus according to an exemplary embodiment as described above, the user may effectively separate or detect the target bioacoustics to be used for diagnosis.

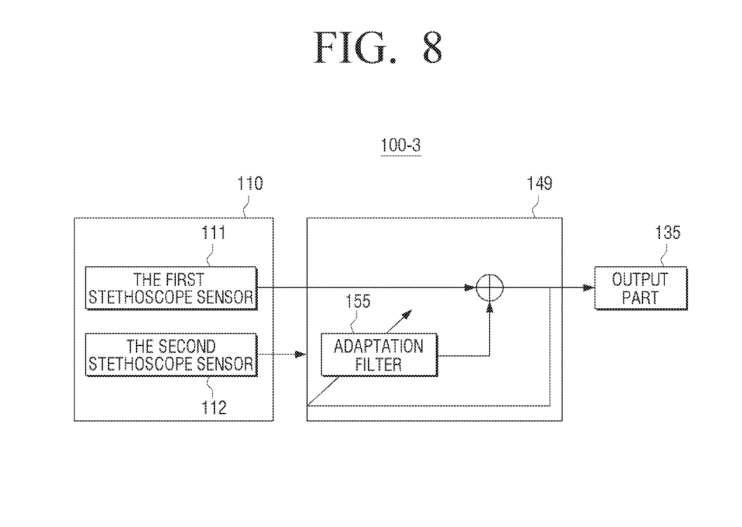

FIG. 8 is a block diagram of a configuration of an electronic stethoscope apparatus 100-3 according to another exemplary embodiment.

The stethoscope apparatus 100-3 includes a bioacoustics sensing part 110, which includes a first stethoscope sensor 111 and a second stethoscope sensor 112, a detecting part 149, and an output part 135.

The bioacoustics sensing part 110 uses a plurality of stethoscope sensors to sense a reference bioacoustics and a synthesis bioacoustics including the reference bioacoustics and a target bioacoustics. In particular, the bioacoustics sensing part 110, as illustrated in FIG. 8, includes the first 111 and the second 112 stethoscope sensor. The bioacoustics sensing part 110 senses the synthesis bioacoustics, which are synthesized with the reference bioacoustics and the target bioacoustics, through the first stethoscope sensor 111, and senses the reference bioacoustics through the second stethoscope sensor 112. The method in which the bioacoustics sensing part 110 uses a plurality of the stethoscope sensors for signal processing has already been described and thus the description thereof will be omitted.

The detecting part 149 uses an adaptation filter 155 to remove the reference bioacoustics sensed by the second stethoscope sensor 112 from the synthesis bioacoustics sensed by the first stethoscope sensor 111 and detect the target bioacoustics. Specifically, if the synthesis bioacoustics signal measured from the first stethoscope sensor 111 is input into the detecting part 149 in the state where the reference bioacoustics is mixed in the synthesis bioacoustics signal, the adaptation filter 155 of the detecting part detects an output value of the detecting part 149 and changes a coefficient of the adaptation filter 155 based on the output value to perform feedback. In this manner, the adaptation filter 155 may filter the reference bioacoustics signal from the measured synthesis bioacoustics signal and detects the target bioacoustics.

The output part 135 outputs the target bioacoustics detected by the detecting part 149. The output part 135 may output the target bioacoustics in an audio form, however, this is only an example and the target bioacoustics may also be output in any other forms, e.g., a video form.

As described above, by using the apparatuses and methods according to the exemplary embodiments, bioacoustics with noise may be accurately measured. For example, a pulse sound of a mother may be mixed as noise with a heart sound of a fetus when measuring the heart sound of the fetus. In other words, the synthesis bioacoustics synthesized with the heart sound of a fetus and the pulse sound of a mother may be obtained by contacting the first stethoscope sensor 111 on the mother's abdomen. Also, the pulse sound of a mother can be obtained as a reference bioacoustics by contacting the second stethoscope sensor 112 on radial artery in which a pulse is detected. With the method described above, the user may measure the heart sound of a fetus more clearly by removing the pulse sound of a mother which is a reference bioacoustics.

The stethoscope apparatus according various exemplary embodiments enables automatic disease diagnosis through outputted stethoscope signals. In addition, the accuracy of diagnosis can be higher in a combination of different medical technologies, as described below.

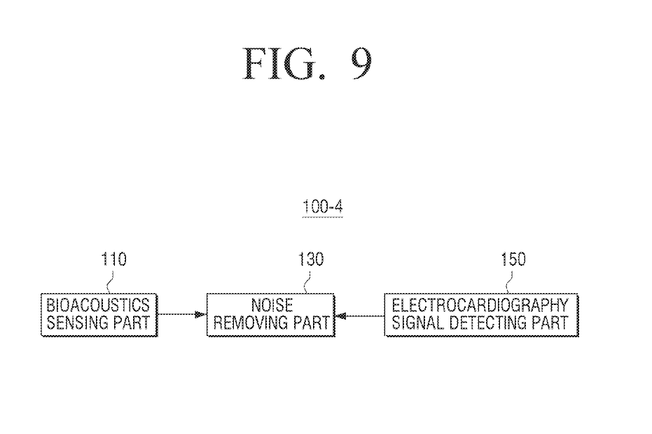

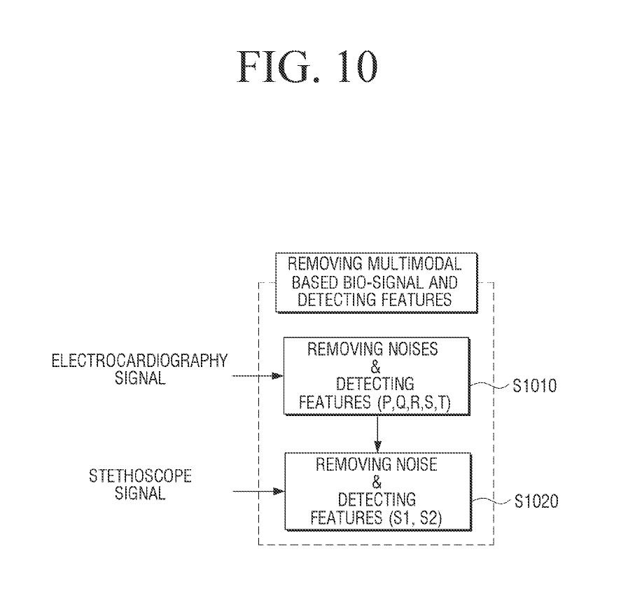

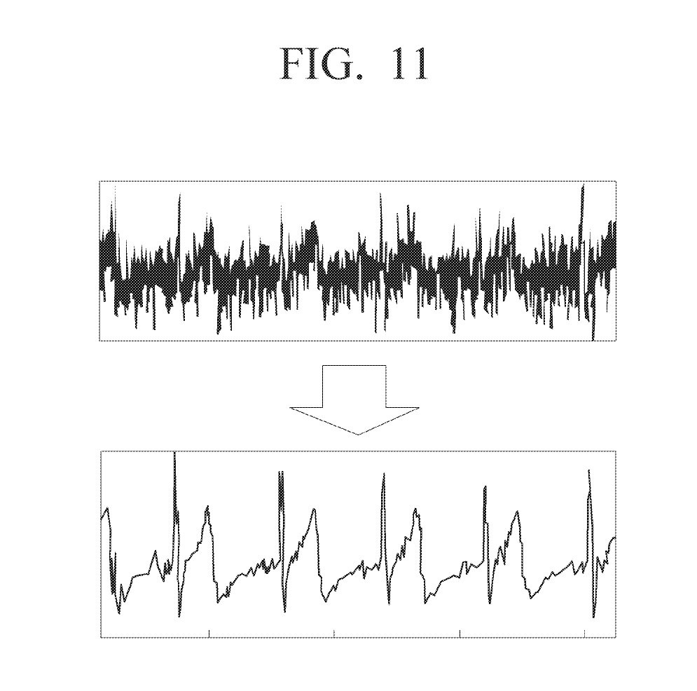

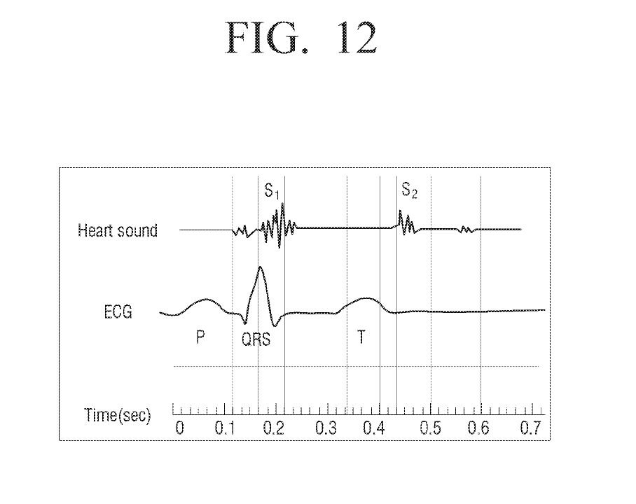

FIG. 9 is a block diagram illustrating a block diagram illustrating a configuration of an electronic stethoscope apparatus 100-4 according to another exemplary embodiment. FIG. 10 is a reference diagram schematically illustrating an operation of the electronic stethoscope apparatus 100-4 according to another exemplary embodiment. FIG. 11 is a drawing illustrating a wave form of an electrocardiography signal. FIG. 12 is a drawing illustrating a location correlation between a stethoscope signal and an electrocardiography signal.

Referring to FIG. 9, the stethoscope apparatus 100-4 according to another exemplary embodiment includes a bioacoustics sensing part 110, an electrocardiography signal detecting part 150, and a noise removing part 130.

The function of the bioacoustics sensing part 110 is as described above, and thus description thereof is omitted.

The electrocardiography signal detecting part 150 is a configuration for detecting an electrocardiography signal. Specifically, the electrocardiography signal detecting part 150 detects a subtle electronic signal sensed from skin through a pair of electrodes attached to the skin when a cardiac muscle cell depolarizes at every heartbeat. At a resting phase, each cardiac muscle cell has a negative charge, which is called a membrane potential. These negative charges are decreased due to the inflow of cations such as Na+ and Ca++, and thus the depolarization occurs and the heart contracts. During each heartbeat, the heart provides an orderly depolarization wave form spreading out from the signal coming out from a sinoatrial node to whole ventricle. A wave form of a small voltage sensed by a pair of electrodes can be expressed on display as a curve.

Within one cycle of the electrocardiography signal, a P wave, a Q wave, an R wave, an S wave, and a T wave are generally generated consecutively as illustrated in FIG. 11.

The P wave indicates the contraction of an atrium and a series of the Q wave, the R wave, and the S wave (or a QRS complex) indicates the contraction of a ventricle, and the T wave is feature of the relaxation of a ventricle.

The electrocardiography signal detecting part 150 detects features of the QRS complex section, the P wave, and the T wave (S1010). The QRS complex section is an electrocardiography signal section between a starting point and an ending point of the QRS complex, in which a point where the size of the an electrocardiography signal (i.e., a voltage on a y-axis) is at the peak is a center point. During the process, the noise of an electrocardiography signal is removed as shown in FIG. 10. This is distinguished from the operation of the noise removing part 130, which is described later.

The noise removing part 130 uses the detected electrocardiography signal to estimate the location of the heart sound from the sensed bioacoustics. Then, the noise removing part 130 removes the noise from the sensed bioacoustics (S 1020)

The noise removing part 130 may remove the noise according to the signal feature of the bioacoustics and a respiration signal. Generally, a heart sound has a feature of an impulse signal, whereas a breathing or noise sound has a feature of a white noise. In addition, a heart sound and a respiration signal have different frequency bands, and therefore, it is possible to detect a heart sound using a frequency filter. However, the noise may not be removed completely when detecting a heart sound, and thus, it is desirable to locate a heart sound more precisely because disease can be diagnosed according to the location of noise generated based on the location of a heart sound.

It is possible to diagnose a disease using the above mentioned signal features of a respiration signal. A normal respiration signal has the feature of white noise and the size of the noise decreases in a higher frequency. On the other hand, an abnormal respiration signal includes crackle, wheezing, stridor, pleural rub, etc., and has a different frequency feature from that of the normal respiration signal in case of diseases related to respiration organs.

As illustrated in FIG. 12, there is a correlation between the location of the stethoscope signal and the electrocardiography signal. That is, the heart sound of the stethoscope signal is located at the end part of the QRS section of the electrocardiography signal. Therefore, the noise removing part 130 uses the electrocardiography signal to locate the heart sound of the stethoscope signal. In particular, when a lot of noise is included in the stethoscope signal, it may be difficult to locate the location of the heart sound based on the stethoscope signal only. In this case, the location of the heart sound of the stethoscope signal may be located using the electrocardiography signal.

The electronic stethoscope device 100-3 according to another exemplary embodiment may remove the noise by detecting the features of the electrocardiography signal through the electrocardiography signal detecting part 150. Then, the information is used to detect features of the stethoscope signal. That is, the features of the stethoscope signal may be used to remove the noise from the stethoscope signal and detect accurate features of a heart sound or a pulmonary sound.

When auscultating a respiration signal, the noise removing part 130 filters the heart sound from the detected bioacoustics after estimating the location of the heart sound through the features of the stethoscope signal.

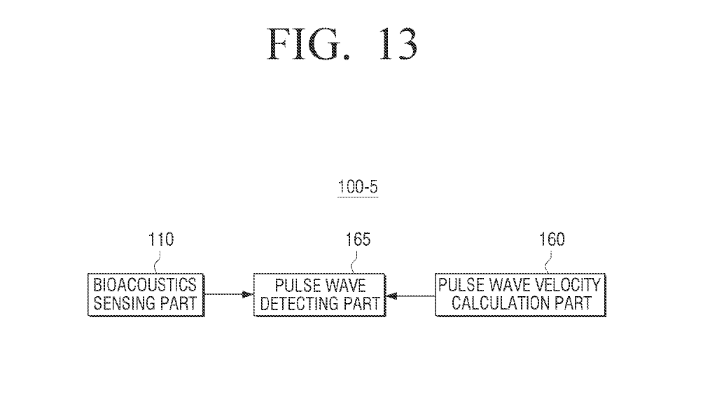

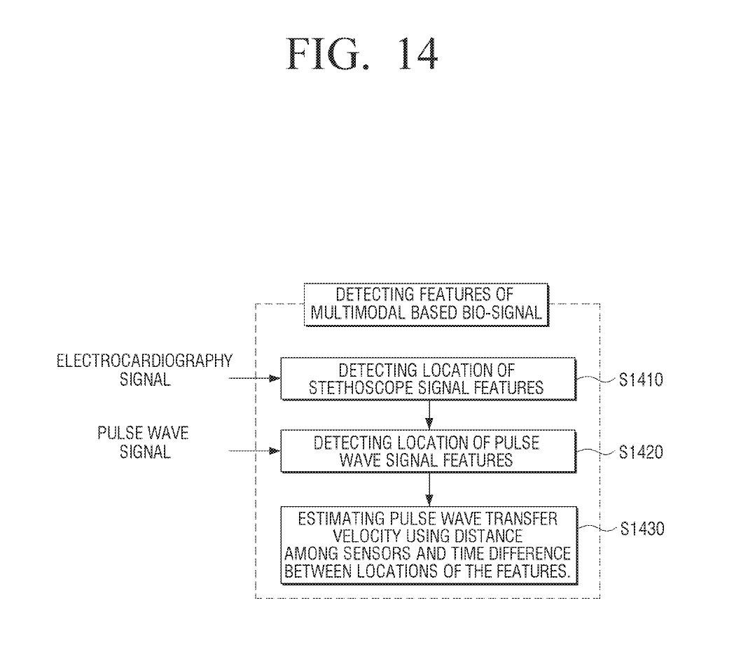

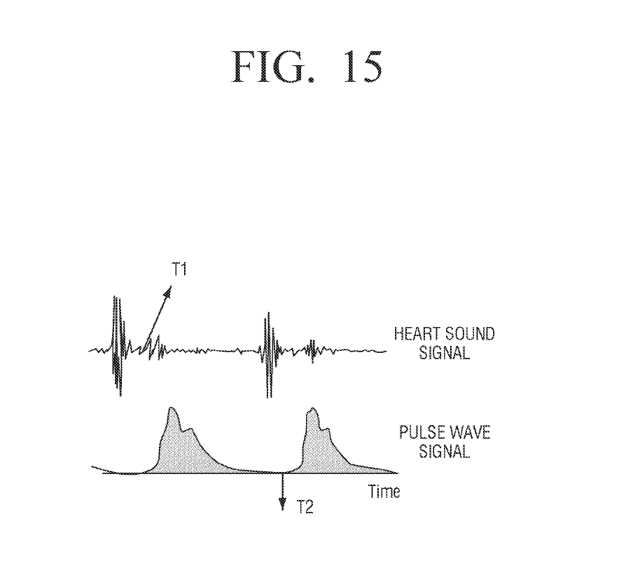

FIG. 13 is a block diagram illustrating a configuration of an electronic stethoscope apparatus 100-5 according to another exemplary embodiment. FIG. 14 is a reference diagram schematically illustrating an operation of an electronic stethoscope apparatus 100-5 according to another exemplary embodiment. FIG. 15 is a drawing illustrating wave forms of a heart sound and a pulse wave.

Referring to FIG. 13, the stethoscope apparatus 100-5 includes a bioacoustics sensing part 110, a pulse wave signal detecting part 160, and a pulse wave velocity calculating part 165.

The bioacoustics sensing part 110, as mentioned above, outputs the stethoscope signal. As mentioned above, the noise filtering from the sensed bioacoustics is possible, and features of the stethoscope signal can be detected (S1410).

The pulse wave signal detecting part 160 detects a pulse wave signal. The pulse wave signal detecting part 160 measures the pulse wave generated as a result of the repetition of the contraction and relaxation of a human heart from the bold vessel. The pulse wave signal detecting part 160 is configured with sensor elements having several physical features according to the measuring purpose. For example, a pressure sensor using elements such as a piezo element for generating an output signal according to the pressure may be used. The pulse wave signal detecting part 160 detects the pulse wave signal and calculates the location of the feature thereof (S1420).

The pulse wave velocity calculating part 165 uses the time between a first location of the sensed bioacoustics (or a location at which a feature of the sensed bioacoustics is detected) and a second location of the pulse wave signal (or a location at which a feature of the pulse wave signal is detected) to estimate the pulse wave transfer velocity (S 1430)

Referring to FIG. 15, a start location time (T1) of the heart sound is estimated and a time (T2) of the incisura (v point) location is measured from the pulse wave in an exemplary embodiment. Then, the pulse wave transfer velocity may be estimated by using both a distance between the bioacoustics sensing part 110 and the pulse wave signal detecting part 160 and a time difference between the start location time (T1) of the heart sound and the time (T2) of the incisura (v point) location.

In a case where the pulse wave transfer velocity is lower, the blood vessel can be diagnosed as healthy because the wave of the pulse wave is absorbed and the pulse wave transfer velocity slows down in case of a soft and elastic blood vessel. The pulse wave transfer velocity also slows down when the blood vessel has a greater internal diameter.

The electronic stethoscope apparatus having an ultrasonic detecting means is described below.

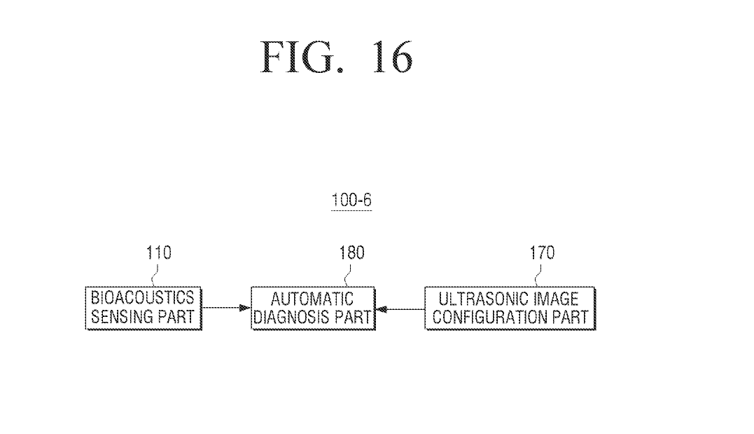

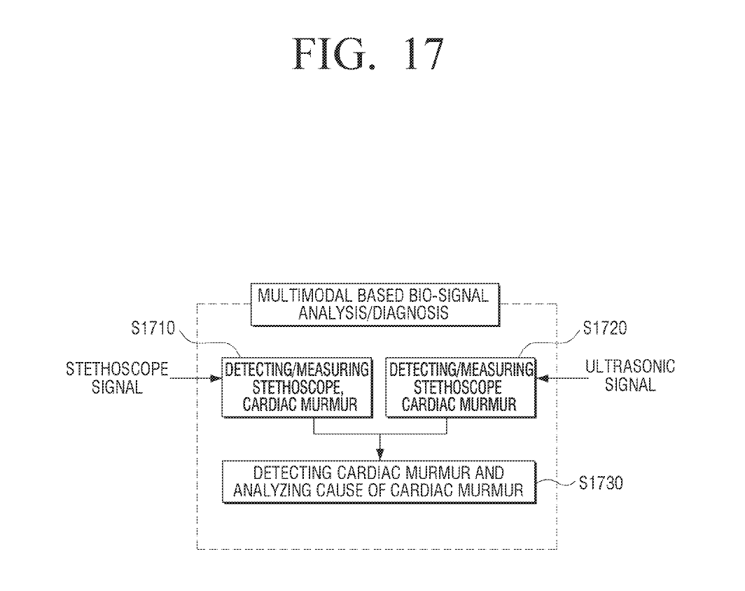

FIG. 16 is a block diagram illustrating an operation of an electronic stethoscope apparatus 100-6 according to another exemplary embodiment. FIG. 17 is a reference diagram schematically illustrating an operation of an electronic stethoscope apparatus 100-6 according to another exemplary embodiment.

Referring to FIG. 16, the electronic stethoscope apparatus 100-6 includes a bioacoustics sensing part 110, an ultrasonic image configuration part 170, and an automatic diagnosis part 180.

The bioacoustics sensing part 110 outputs the stethoscope signal (S1710). As mentioned above, the noise filtering from the sensed bioacoustics is possible.

The ultrasonic image configuration part (or an ultrasonic image generator) 170 irradiates an ultrasonic signal onto a body part and senses an ultrasonic echo signal reflected from the body part. For this, the ultrasonic image configuration part 170 includes a probe including a transducer and a vibrator, and a tuning coil for sensing the ultrasonic echo signal. In addition, the ultrasonic image configuration part 170 includes an A/D converter for processing a received signal and a signal processing part.

The ultrasonic image configuration part 170 receives the ultrasonic signal reflected from the body part and may generate a B-mode (brightness mode) image having a black-and-white signal or a C-mode (color mode or color Doppler) image.

The B-mode image is an image which represents the body part where the ultrasonic is irradiated in black and white by using the ultrasonic echo signal reflected from the body part. In a coordinate system comprising a horizontal axis represented by the distance to the body part and a vertical axis represented by the amplitude of the reflected echo, the amplitude can be represented by the brightness of a coordinate. The B-mode image can be configured in black and white in this manner.

On the other hand, the C-mode image is an image which represents the body part where ultrasonic is irradiated in color by using the reflected ultrasonic echo signal. In a case where the ultrasonic echo signal is received and then a frequency deviation occurs by the Doppler Effect, the ultrasonic image configuration part 170 may measure the velocity of a blood flow by calculating the frequency deviation. Then, the C-mode image can be configured using the same.

The automatic diagnosis part (or automatic diagnoser) 180 performs automatic diagnosis by using the ultrasonic image and the bioacoustics signal. Specifically, the automatic diagnosis part 180 performs automatic diagnosis by analyzing the heart sound and the ultrasonic image to determine a cause of heart sound (S 1730). For this, parameters on various diseases are stored in advance and managed, and the disease matching the parameters of ultrasonic image and bioacoustics signal is searched. In an exemplary embodiment, the ultrasonic sound that is converted from the ultrasonic image and/or the ultrasonic image can be used.

A remote diagnosis system 1000 according to various exemplary embodiments is described below.

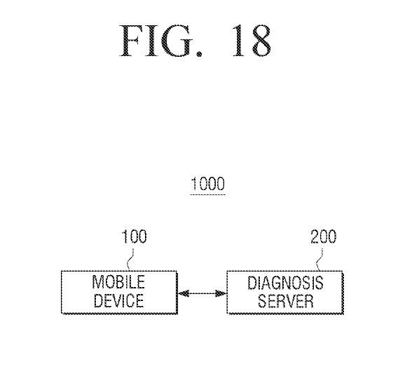

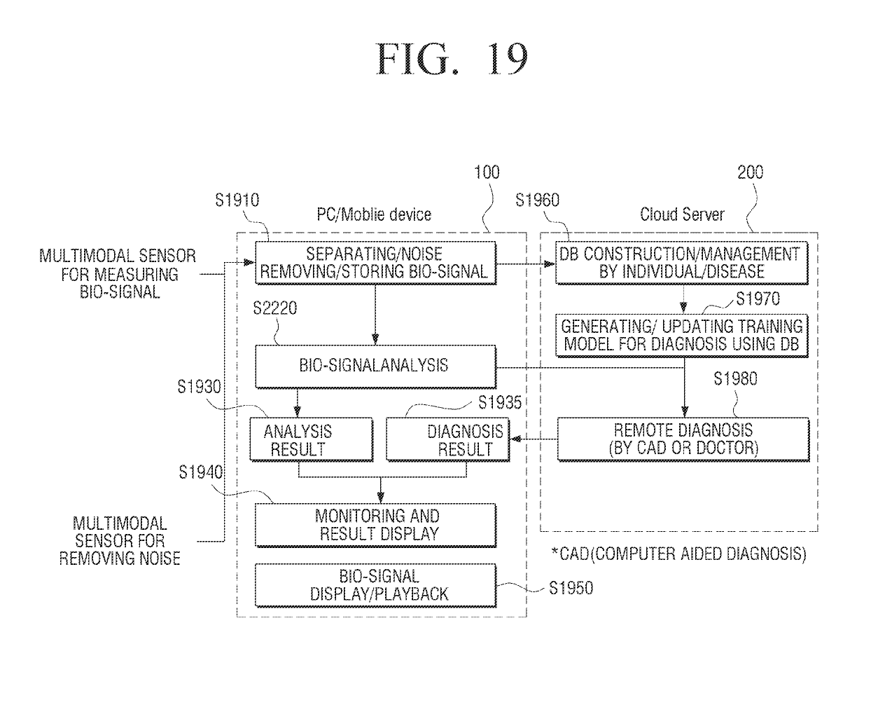

FIG. 18 is a block diagram illustrating a configuration of a remote diagnosis system 1000 according to an exemplary embodiment. FIG. 19 is a flowchart illustrating a remote diagnosis method according to an exemplary embodiment.

Referring to FIG. 18, the remote diagnosis system 1000 includes a mobile device 100 and a diagnosis server 200.

The mobile device 100 detects or measures various bio-signals (S1910). The mobile device 100 removes noise from a bio-signal by using the above mentioned various methods and stores the bio-signal from which noise has been removed. Then, the mobile device 100 analyzes the bio-signal (S1920). The result of the diagnosis can be displayed (S1930). In an exemplary embodiment, the result can be displayed with the result of the diagnosis by the diagnosis server 200 (S1940). The diagnosis server 200 generates and manages a database (DB) according to a user and/or disease based on a bio-signal received from the mobile device 100 (S1960). Then, the diagnosis server 200 generates and updates a training model for diagnosis using the DB (S1970). The training model is used in automatic diagnosis.

The remote diagnosis can be performed by a doctor, or by an automatic diagnosis of the diagnosis server 200. The result of the diagnosis is transmitted to the mobile device 100 (S1935). The mobile device 100 displays the result of the diagnosis.

As mentioned above, an abnormal respiration signal can be detected through a stethoscope signal. Therefore, it is possible to diagnosis diseases such as bronchitis, pulmonary edema, cardiac insufficiency, pneumonia, pulmonary infarction, and asthma etc. In addition, it is possible to diagnose cardiac murmur, arrhythmia, heart rate, etc. that is related to heart diseases.

By using electrocardiography, it is possible to diagnose atrial fibrillation, atrial flutter, ventricular tachycardia, myocardial infarction, ischemic heart disease, and valvular heart disease which may cause abnormality of blood flow through heart rate and arrhythmia.

Arrhythmia, a heart rate, heart valve stenosis, obstruction, etc. maybe diagnosed through an ultrasonic image or sound, and it is also possible to diagnose the condition of the blood vessel. That is, valvular heart disease, stenosis and obstruction causing abnormality in a blood flow can be diagnosed.

Arrhythmia and a heart rate can be diagnosed through the pulse wave, and it is possible to diagnose vein aging, vein elasticity, a blood circulation age, an artery disorder, and a peripheral vascular disorder, etc.

The automatic diagnosis method according to various exemplary embodiments is described below.

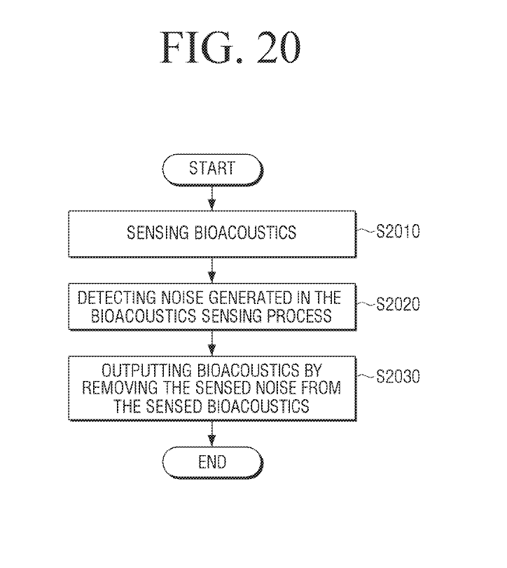

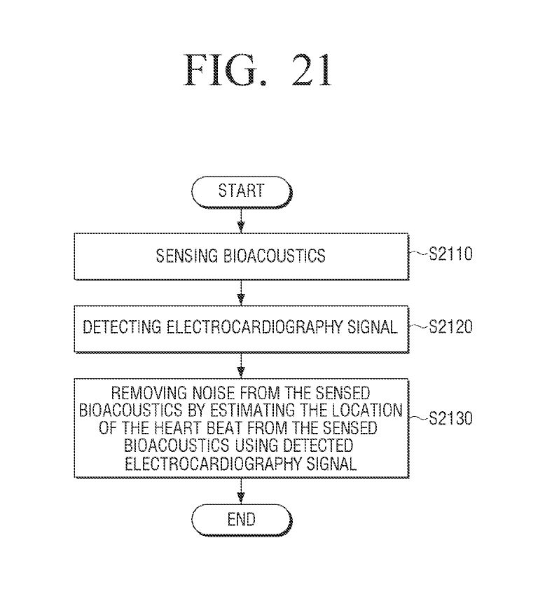

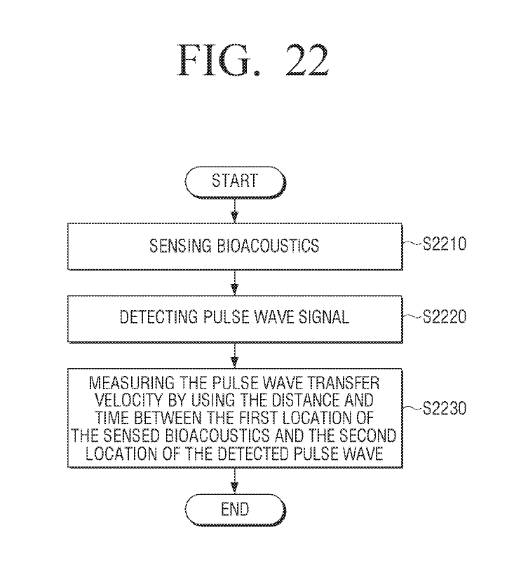

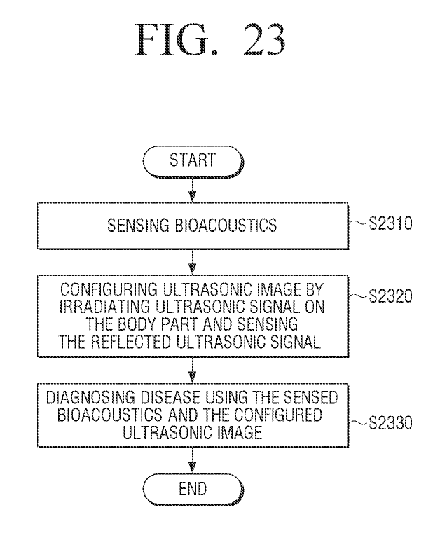

FIGS. 20, 21, 22, and 23 are flowcharts of an automatic diagnostic method according to various exemplary embodiments.

The automatic diagnosis method according to an exemplary embodiment includes sensing bioacoustics (S2010), sensing noise generated in the bioacoustics sensing process (S2020), and outputting bioacoustics by removing the sensed noise from the sensed bioacoustics (S2030).

The noise sensing operation may perform using a microphone.

In addition, the microphone is mounted on the chest piece of the electronic stethoscope apparatus, and the noise removing operation may be performed to output the sensed bioacoustics by filtering the frequency of the noised signal sensed through the microphone.

Furthermore, in the noise sensing operation, a movement sensor for sensing movement of the electronic stethoscope apparatus may be used to sense the noise, and in the noise removing operation, the sensing value which is output from the movement sensor may be used to calculate the noise signal and to output the sensed bioacoustics by filtering the frequency of the noise signal.

The automatic diagnosis method according to another exemplary embodiment includes sensing bioacoustics (S2110), detecting electrocardiography signal (S2120), and estimating the location of the heart sound from the sensed bioacoustics by using the detected electrocardiography signal and of removing the noise from the sensed bioacoustics (S2130).

The electrocardiography signal detecting operation detects the features of the detected electrocardiography signal, and the noise removing operation uses the feature of the detected electrocardiography signal to estimate the location of a heart sound from the sensed bioacoustics.

The automatic diagnosis method according to another exemplary embodiment includes sensing bioacoustics (S2210), detecting a pulse wave signal (S2220), and measuring the pulse wave transfer velocity by using the distance between sensors (e.g., the bioacoustics sensing part 110 and the pulse wave signal detecting part 160) and a time difference between the first location of the sensed bioacoustics (or a location at which a feature of the sensed bioacoustics is detected) and the second location of the detected pulse wave signal (or a location at which a feature of the detected pulse wave signal) (S2230).

The automatic diagnosis method according to another exemplary embodiment includes sensing bioacoustics (S2310), configuring an ultrasonic image by irradiating an ultrasonic signal onto a body part and sensing the reflected ultrasonic signal (S2320), and diagnosing disease by using the sensed bioacoustics and the configured ultrasonic image (S2330).

According to various exemplary embodiments as described above, an electronic stethoscope apparatus for removing noise properly may be provided.

In addition, the exemplary embodiments provide techniques of estimating bioacoustics and diagnosing diseases by considering information from other devices.

The automatic diagnosis method according to exemplary embodiments may be embodied as an algorithm or a computer program and may be stored on a computer-readable recording medium as computer readable codes or program commands executable by a processor. Examples of the computer-readable recording medium include a compact disc (CD), a digital versatile disc (DVD), a hard drive, a Blu-ray disc, a universal serial bus (USB), a memory card, a read-only memory (ROM), a floppy disk, a CD-ROMs, and the like. The computer-readable recording medium may also be distributed over network-coupled computer systems so that the computer readable code is stored and executed in a distributed fashion. The recoding medium may be read by a computer, stored in a memory, and executed by the processor.

At least one of the components, elements or units represented by a block as illustrated in the drawings may be embodied as various numbers of hardware, software and/or firmware structures that execute respective functions described above, according to an exemplary embodiment. For example, at least one of these components, elements or units may use a direct circuit structure, such as a memory, processing, logic, a look-up table, etc. that may execute the respective functions through controls of one or more microprocessors or other control apparatuses. Also, at least one of these components, elements or units may be specifically embodied by a module, a program, or a part of code, which contains one or more executable instructions for performing specified logic functions. Also, at least one of these components, elements or units may further include a processor such as a central processing unit (CPU) that performs the respective functions, a microprocessor, or the like. Further, although a bus is not illustrated in the above block diagrams, communication between the components, elements or units may be performed through the bus. Functional aspects of the above exemplary embodiments may be implemented in algorithms that execute on one or more processors. Furthermore, the components, elements or units represented by a block or processing steps may employ any number of related art techniques for electronics configuration, signal processing and/or control, data processing and the like.

Although a few embodiments have been shown and described, it would be appreciated by those skilled in the art that changes may be made in the exemplary embodiments without departing from the principles and spirit of the disclosure, the scope of which is defined in the claims and their equivalents.

1. An electronic stethoscope apparatus comprising:
a bioacoustics sensor configured to sense bioacoustics of an object;
a noise sensor configured to sense a noise in the sensed bioacoustics; and
a noise remover configured to remove the sensed noise and output the sensed bioacoustics from which the noise has been removed.

2. The electronic stethoscope apparatus according to claim 1, wherein the bioacoustics sensor comprises a microphone.

3. The electronic stethoscope apparatus according to claim 1, wherein the noise sensor comprises a microphone and mounted on a chest piece of the electronic stethoscope apparatus; and
the noise remover is configured to output the sensed bioacoustics by filtering a frequency of a noise signal sensed by the microphone.

4. The electronic stethoscope apparatus according to claim 1, wherein the noise sensor comprises a movement sensor configured to sense movement of the electronic stethoscope apparatus, and the noise remover is configured to output the sensed bioacoustics by filtering a frequency of a noise signal sensed by the movement sensor.

5. An electronic stethoscope apparatus comprising:
a bioacoustics sensor configured to sense bioacoustics of an object;
an electrocardiography_(ECG) detector configured to detect an electrocardiography signal from the object; and
a noise remover configured to estimate a position of a heart sound from the sensed bioacoustics by using the detected electrocardiography signal and remove a corresponding noise from the sensed bioacoustics based on the position of the heart sound.

6. The electronic stethoscope apparatus according to claim 5, wherein the bioacoustics sensor is configured to extract a feature of the detected electrocardiography (ECG) signal;, and
the noise remover is configured to estimate the position of the heart sound from the sensed bioacoustics by using the feature of the detected electrocardiography (ECG) signal.

7. An electronic stethoscope apparatus comprising:
a bioacoustics sensor configured to sense bioacoustics of an object;
a pulse wave signal detector configured to detect a pulse wave signal of the object; and
a pulse wave velocity calculator configured to calculate a pulse wave transfer velocity by using a distance between the bioacoustics sensor and the pulse wave signal detector and a time difference between detecting a first position of the sensed bioacoustics and detecting a second position of the detected pulse wave signal.

8. An automatic diagnosis apparatus comprising:
a bioacoustics sensor configured to sense bioacoustics of an object;
an ultrasonic image generator configured to generate an ultrasonic image by irradiating an ultrasonic signal onto the object and sensing the ultrasonic signal that is reflected from the object; and
an automatic diagnoser configured to diagnose a disease by using the sensed bioacoustics and the generated ultrasonic image.

9. An automatic diagnosis method by using an electronic stethoscope apparatus, the automatic diagnosis method comprising:
sensing a bioacoustics of an object;
sensing a noise in the sensed bioacoustics; and
removing the sensed noise and outputting the sensed bioacoustics from which the noise has been removed.

10. The automatic diagnosis method according to claim 9, further comprising:
providing a microphone on a chest piece of the electronic stethoscope apparatus, wherein the sensing the noise is performed by using the microphone.

11. The automatic diagnosis method according to claim 10, wherein
the removing comprises removing the sensed noise from the sensed bioacoustics by filtering a frequency of a noise signal sensed by the microphone.

12. The automatic diagnosis method according to claim 9, further comprising:
providing a movement sensor configured to sense movement of the electronic stethoscope apparatus, wherein the sensing the noise is performed by the movement sensor,
wherein the removing comprises removing the sensed noise from the sensed bioacoustics by filtering a frequency of a noise signal that is sensed based on the movement sensor.

13. An automatic diagnosis method comprising:
sensing bioacoustics of an object;
detecting an electrocardiography signal of the object;
estimating a position of a heart sound from the sensed bioacoustics by using the detecting electrocardiography signal and removing a corresponding noise from the sensed bioacoustics based on the position of the heart sound.

14. The automatic diagnosis method according to claim 13, wherein the detecting comprises extracting a features of the detected electrocardiography signal, and
the estimating comprises estimating the position of the heart sound from the sensed bioacoustics by using the extracted features of the detected electrocardiography signal.

15. An automatic diagnosis method comprising:
sensing bioacoustics of an object;
detecting a pulse wave signal of the object; and
calculating a pulse wave transfer velocity by using a distance in locations of the sensing and the detecting and a time difference between detecting a first position of the sensed bioacoustics and detecting a second position of the detected pulse wave signal.
